(19) United States
(12) Patent Application Publication
(10) Pub. No.: US 2005/0222271 A1
(43) Pub. Date: Oct. 6, 2005

(54) Novel amorphous form of memantine hydrochloride

(76) Inventor: Le Huang, Shang Gao (CN)

Correspondence Address: Hui Min He-Huang, 53 Assabet Drive, Northborough, MA, 01532, US

(21) Appl. No.: 10/815,090

(22) Filed: Mar. 31, 2004

(51) Int. Cl. A61K 31/13

(52) U.S. Cl. 514/662; 564/459

(57) ABSTRACT

The present invention relates to novel amorphous form of 1-amino-3, 5-dimethyltricyclo[3,3,1,1^{3,7}]decane hydrochloride (memantine hydrochloride), to processes for its preparation, to pharmaceutical compositions containing it and to method of treatment using the same. The amorphous form of memantine hydrochloride obtained is a known valuable agent useful in treating Alzheimer's disease or conditions in which N-methyl-D-aspartate (NMDA)-receptor antagonist is implicated.

### FIELD OF THE INVENTION

The present invention is directed to novel amorphous form of memantine hydrochloride. The invention also relates to processes for preparing the amorphous form, to pharmaceutical compositions containing it, and to method of treatment using the same.

### BACKGROUND OF THE INVENTION

Memantine hydrochloride is the common chemical name for 1-amino-3, 5-dimethyltricyclo[3,3,1,1^{3,7}]decane hydrochloride or 1-amino-3,5-dimethyl adamantine (memantine hydrochloride), is represented by the Formula I:

Formula I inert here

Memantine hydrochloride is the first FDA approved member of a new class of Alzheimer drugs—a moderate affinity N-methyl-D-aspartate (NMDA)-receptor antagonist. It produces symptomatic improvements in learning under condition of tonic NMDA receptor activation in Alzheimer's disease. In contrast to first generation therapies, memantine hydrochloride is likely to show neuroprotective effect at concentration used in the treatment of Alzheimer's disease and to slow down disease progression.

Bormann et al. U.S. Pat. No. 5,061,703 discloses that memantine hydrochloride is useful for the prevention and treatment of cerebral ischemia after apoplexy, open-heart surgery, cardiac standill, subarachnoidal hemorrhage, transient cerebro-ischemic attacks, perinatal asphyxia, anoxia, hypoglycemia, apnoca and Alzheimer's disease. U.S. Pat. No. 5,614,560 further discloses a method for reducing non-ischemic NMDA receptor mediated neuronal degeneration in a mammal in disease state.

From a treatise in the publication “Journal of Medicinal Chemistry” 6,6 (1963), pp. 760-763, a method of preparation of 1-amino-3, 5-dimethyl adamantine (memantine) is known. U.S. Pat. No. 4,122,193 discloses a method to prepare memantine hydrochloride by heating 1-chloro-3, 5-dimethyl adamantine and urea at 220° C. The heating was carried out in a closed vessel in an oil bath with a thermostat. After cooling, the reaction product was pulverized and made into a paste with water. The water phase was brought to a pH between 3 and 5 by dropwise addition of concentrated HCl. The acidified water phase was extracted with two ether portions. The water phase was then brought to a pH between 12 and 13 by addition of sodium hydroxide solution. After stirring, the alkaline water phase was extracted with four portions of ether. The combined ether extracts were dried over potassium hydroxide. By bubbling dried hydrogen chloride through the solution, 1-amino-3, 5-dimethyl adamantine chloride (memantine chloride) was obtained. The product in this reference did not melt until 300° C. All of references cited above did not disclose on amorphous forms of memantine hydrochloride.

C.N. Pat. Nos. 1400205 A and 1335299 A disclose an improved method to make memantine chloride by reacting 1-bromo-3, 5-dimethyl adamantine with urea in a polyol solvent (such as HOCH₂CH₂OH), followed by treatment with sodium hydroxide, and acidification with hydrochloric acid. These references also did not disclose on amorphous forms of memantine hydrochloride.

Memantine hydrochloride crystals (melting point is over 300° C., as described in U.S. Pat. No. 4,122,193) and its derivative such as amantadine hydrochloride crystals (melting point is about 360° C., as described in Merck Index, 13^{th}, 389, pp 65) have very high crystal lattice energy so that their crystals have undesirably high melting points, which often have a significant impact on their bioavailability when used as pharmaceutical agents.

It has been disclosed earlier that the amorphous forms in a number of drugs exhibit different dissolution characteristics and in some cases different bioavailabilily patterns compared to crystalline forms [Konne T., Chem Pharm Bull, 38, 2003 (1990)]. For some therapeutic indications one bioavailability pattern may be favored over another. An amorphous form of cefuroxime axietil is good example for exhibiting higher bioavailability than the crystalline forms.

### SUMMARY OF THE INVENTION

We have now surprisingly and unexpectedly discovered that novel amorphous form of memantine hydrochloride can be prepared.

In one aspect, the present invention relates to novel amorphous form of 1-amino-3, 5-dimethyltricyclo[3,3,1,1^{3,7}]decane hydrochloride (memantine hydrochloride), including anhydrous amorphous form and amorphous hydrate of memantine hydrochlordie.

In another aspect, the present invention relates to a process for preparing amorphous form of memantine hydrochloride, including the steps of dissolving memantine hydrochloride in a solvent to form a solution and lyophilizing the solvent from the solution to afford amorphous form of memantine hydrochloride.

In another aspect, the present invention relates to a process for preparing amorphous form of memantine hydrochloride, including the steps of dissolving memantine hydrochloride in water to form a solution and lyophilizing the water from the solution to afford amorphous form of memantine hydrochloride.

In another aspect, the present invention relates to a process for preparing amorphous form of memantine hydrochloride, including the steps of dissolving memantine hydrochloride in a solvent to form a solution and distilling the solvent from the solution to afford amorphous form of memantine hydrochloride.

In another aspect, the present invention relates to a process for preparing amorphous form of memantine hydrochloride, including the steps of dissolving memantine hydrochloride in methanol or ethanol to form a solution and distilling the methanol or ethanol solvent from the solution to afford amorphous form of memantine hydrochloride.

Another aspect of the present invention is a pharmaceutical composition for administering effective amount novel amorphous form of memantine hydrochloride in unit dosage form.

According to a further aspect of the invention is a method for prevention and treatment of cerebral ischemia after apoplexy, open-heart surgery, cardiac standill, subarachnoidal hemorrhage, transient cerebro-ischemic attacks, perinatal asphyxia, anoxia, hypoglycemia, apnoca and Alzheimer's disease, with a medicament made by using an effective amount of novel amorphous form of memantine hydrochloride in unit dosage form.

### BRIEF DESCRIPTION OF THE ACCOMPANYING DRAWINGS

FIG. 1 is X-ray powder diffraction pattern of novel amorphous form of memantine hydrochloride.

### DETAILED DESCRIPTION OF THE INVENTION

As previously described, memantine hydrochloride is a moderate affinity N-methyl-D-aspartate (NMDA)-receptor antagonist, and is useful for the prevention and treatment of moderate to severe Alzheimer's disease.

In one embodiment, this invention provides novel amorphous form of 1-amino-3, 5-dimethyltricyclo[3,3,1,1^{3,7}]decane hydrochloride (memantine hydrochloride).

In another embodiment, the present invention further provides a process of preparing a novel amorphous form of 1-amino-3, 5-dimethyltricyclo[3,3,1,1^{3,7}]decane hydrochloride (memantine hydrochloride).

“Amorphous” means a solid without long-range crystalline order. Amorphous form of memantine hydrochloride in accordance with the invention preferably contains less than about 10% crystalline forms of memantine hydrochloride, and more preferably is essentially free of crystalline forms of memantine hydrochloride. “Essentially free of crystalline forms of memantine hydrochloride” means that no crystalline forms of memantine hydrochloride can be detected within the limits of a powder X-ray diffractometer.

In another embodiment, the present invention provides processes for making amorphous form of memantine hydrochloride by either a lyophilization process or a distillation process. The starting material for either process can be crude or pure memantine hydrochloride obtained by any method, such as the methods described in the patents previously discussed, i.e., U.S. Pat. No. 4,122,193. The starting material for either process can also be crystalline forms of memantine hydrochloride or a mixture of amorphous and crystalline forms of memantine hydrochloride obtained by any method.

In one embodiment, the present invention provides a lyophilizing (freeze drying) process for preparing amorphous forms of memantine hydrochloride, including the steps of dissolving memantine hydrochloride in a solvent to form a solution and lyophilizing the solvent from the solution to afford amorphous forms of memantine hydrochloride.

In a first step of the lyophilization process, memantine hydrochloride is preferably dissolved in an aqueous (prepared with water) solvent, more preferably dissolved in an aqueous alcohol solvent, and most preferably dissolved in water to form a solution.

In particular, memantine hydrochloride is soluble in water, allowing the complete dissolution of memantine hydrochloride at room temperature at a concentration of 50 mg per milliliter (ml). The use of a relatively concentrated solution, e.g. about 50 mg per ml is therefore preferred.

In a second and preferred step of the lyophilization process, a solution of memantine hydrochloride in a solvent is lyophilized to leave a solid residue containing memantine hydrochloride in an amorphous state. In this invention, the lyophilization step is performed in two stages: freezing and drying.

In the first stage of lyophilization, the temperature of the solution is decreased until the solution is completely frozen, typically to temperatures as low as minus 50° C., and below, to produce a frozen mixture. Such cooling allows the solute and solvent to separate into separate solid phases. Usually, phase separation will yield a solute in an amorphous state, but may also yield crystalline, microcrystalline or their mixtures. Preferably in this invention, cooling is performed rapidly so that the formation of solute crystals is inhibited, and only amorphous material is formed. More preferably, the solution is cooled using liquid nitrogen with swirling of the vessel containing the solution to coat the wall of the vessel and accelerate freezing. Once the solution has been completely frozen, it is then possible to remove the separated solvent from the frozen mixture by warming up the contents slowly so that the solvent leaves the frozen mixture through sublimation.

The drying stage is preferably conducted under vacuum so that the frozen solvent will vaporize without melting. Heat is applied to transform the frozen solvent into solvent vapor. This vapor migrates through the frozen mixture and escapes into the evacuated space outside of the frozen mixture. The vapor is re-condensed on a refrigerated surface, and turns into a liquid in condenser. The condenser is maintained at a temperature below that of the frozen mixture to drive the drying process.

When the solvent is water, typical lyophilization conditions for producing amorphous form of memantine hydrochloride include that the temperature of the frozen mixture is from about −50° C. to about 0° C. before vacuum is applied. The vacuum is typically about 0.05 mm Hg or less, more preferably about 0.01 mm Hg or less and the temperature of the frozen mixture is from about −50° C. to about 20° C. during the drying stage. The drying time using these conditions and standard equipment is dependent on the amount and the nature of solute and solvent used. The drying time is from about 24 hours to about 96 hours for about a 50 g sample of memantine hydrochloride dissolved in water.

In another embodiment, the present invention also provides a distillation process for preparing amorphous form of memantine hydrochloride, including the steps of dissolving memantine hydrochloride in a solvent to form a solution and distilling the solvent from the solution to dryness to afford amorphous form of memantine hydrochloride.

In a first step of the distillation process, memantine hydrochloride is preferably dissolved in an aqueous solvent; more preferably dissolved in a straight or branched chain C₁-C₄ alcohol solvent, and most preferably dissolved in methanol or ethanol to form a solution. Memantine hydrochloride is soluble in methanol or ethanol, allowing the complete dissolution of memantine hydrochloride at ambient temperature.

In particular, memantine hydrochloride is soluble in methanol, allowing the complete dissolution of memantine hydrochloride in methanol at ambient temperature with a concentration of 30 mg per milliliter (ml). The use of a relatively concentrated solution, e.g. about 30 mg per ml is therefore preferred for methanol.

In particular, memantine hydrochloride is soluble in ethanol, allowing the complete dissolution of memantine hydrochloride in ethanol at ambient temperature with a concentration of 20 mg per ml. The use of a relatively concentrated solution, e.g. about 20 mg per ml is therefore preferred for ethanol.

In a second step of the distillation process, using conventional distillation methods, the solvent is removed from the solution to dryness, thereby leaving a solid residue containing amorphous memantine hydrochloride.

The distillation process can be preformed at atmospheric pressure or reduced pressure. Preferably the solvent is removed at a pressure of about 760 mm Hg or less, more preferably at about 400 mm Hg or less, more preferably at about 80 mm Hg or less, and most preferably from about 30 to about 80 mm Hg.

The straight or branched chain C₁-C₄ alcohol solvents are selected from methanol, ethanol, n-propanol, isopropanol or branched-chain butanols. It is preferred to use methanol or ethanol, or a mixture of methanol and ethanol. The process may also be carried out by using a mixture of two or more other alcohol solvents.

The novel amorphous forms of memantine hydrochloride obtained in above procedures can be anhydrous amorphous and amorphous hydrate. The current invention intends to cover both anhydrous and hydrate amorphous forms of memantine hydrochloride.

It has been unexpectedly found that uniformly anhydrous or hydrate amorphous forms of memantine hydrochloride can be obtained in simple and reproducible processes as described above.

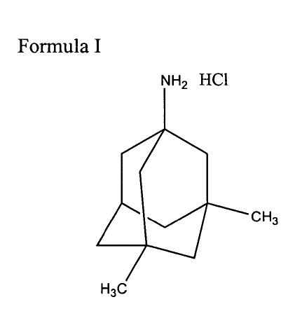

Amorphous form of memantine hydrochloride prepared according to the processes of the present invention may be characterized by its x-ray powder diffration pattern, as shown in the accompanied drawing of FIG. 1. The X-ray powder diffraction pattern (FIG. 1) shows no peaks which are characteristic of amorphous form of memantine hydrochloride, thus demonstrating the amorphous nature of the product.

Another embodiment of the present invention is a pharmaceutical composition for administering effective amount of novel amorphous form of memantine hydrochloride in unit dosage forms.

The unit dosage forms can be administered in a wide variety of oral and parenteral dosage forms. Thus, the compound of the present invention can be administered by injection, that is, intravenously, intramuscularly, intracutaneously, subcutaneously, intraduodenally, or intraperitoneally. Also, the amorphous form of memantine hydrochloride of the present invention can be administered by inhalation, for example, intranasally. Additionally, the compound of the present invention can be administered transdermally. It will be obvious to those skilled in the art that the following dosage forms may comprise as the active component, either amorphous form of memantine hydrochloride, or a corresponding pharmaceutically acceptable salt of a compound of the present invention.

For preparing pharmaceutical compositions from amorphous form of memantine hydrochloride of the present invention, pharmaceutically acceptable carriers can be either solid or liquid.

Solid form compositions include powders, tablets, pills, capsules, cachets, suppositories, and dispersible granules. A solid carrier can be one or more substances that may also act as diluents, flavoring agents, solubilizers, lubricants, suspending agents, binders, preservatives, tablet disintegrating agents, or an encapsulating material.

In powders, the carrier is a finely divided solid that is in a mixture with the finely divided active component. In tablets, the active component is mixed with the carrier having the necessary binding properties in suitable proportions and compacted in the shape and size desired.

The powders and tablets preferably contain from one or ten to about seventy percent of the active compound. Suitable carriers are magnesium carbonate, magnesium stearate, talc, sugar or lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material as a carrier providing a capsule in which the active component, with or without other carriers, is surrounded by a carrier, which is thus in association with it. Similarly, cachets and lozenges are included. Tablets, powders, capsules, pills, cachets, and lozenges can be used as solid dosage forms suitable for oral administration.

For preparing suppositories, a low melting wax, such as a mixture of fatty acid glycerides or cocoa butter, is first melted and the active component is dispersed homogeneously therein, as by stirring. The molten homogenous mixture is then poured into convenient sized molds, allowed to cool, and thereby to solidify.

Liquid form preparations include solutions, suspensions, retention enemas, and emulsions, for example water or water propylene glycol solutions. For parenteral injection, liquid preparations can be formulated in solution in aqueous polyethylene glycol solution.

Aqueous solutions suitable for oral use can be prepared by dissolving the active component in water and adding suitable colorants, flavors, stabilizing, and thickening agents as desired.

Also included are solid form compositions that are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

The pharmaceutical composition is preferably in unit dosage form. In such form, the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

The quantity of active component in a unit dose preparation may be varied or adjusted from 0.5 mg to 50 mg, preferably 2 mg to 20 mg according to the particular application and the potency of the active component. The composition can, if desired, also contain other compatible therapeutic agents. However, the optimum dosage for the individual subject being treated will be determined by the person responsible for treatment, generally smaller doses being administered initially and thereafter increments made to determine the most suitable dosage.

According to a further embodiment of the invention is a method of treating a Alzheimer's disease condition, which comprises administering to warm-blooded mammal, particularly a human, and effective amount of an amorphous form of memantine hydrochloride. As a N-methyl-D-aspartate (NMDA)-receptor antagonist, novel amorphous form of memantine hydrochloride is a useful agent in the prevention and the treatment of cerebral ischemia after apoplexy, open-heart surgery, cardiac standill, subarachnoidal hemorrhage, transient cerebro-ischemic attacks, perinatal asphyxia, anoxia, hypoglycemia, apnoca and Alzheimer's disease or conditions in which N-methyl-D-aspartate (NMDA)-receptor antagonist is implicated, including a long-term non-ischemic neurodegenerative disease as disclosed in U.S. Pat. No. 5,614,560.

In therapeutic use as N-methyl-D-aspartate (NMDA)-receptor antagonist for treating Alzheimer's disease, the amorphous form of memantine hydrochloride utilized in the pharmaceutical method of this invention is administered at the initial dosage of about 0.5 mg to about 50 mg daily. A daily dose range of about 5 mg to about 20 mg is preferred. The dosages, however, may be varied depending upon the requirements of the patient, the severity of the condition being treated and the compound being employed. Determination of the proper dosage for a particular situation is within the skill of the art. Generally, treatment is initiated with smaller dosages which are less than the optimum dose of the compound. Thereafter, the dosage is increased by small increments until the optimum effect under the circumstance is reached. For convenience, the total daily dosage may be divided and administered in portions during the day if desired.

While the present invention has been described in terms of its specific embodiments, certain modifications and equivalents will be apparent to those skilled in the art and are intended to be included within the scope of the present invention.

The following examples are provided to illustrate specific embodiments of the present invention. They are not intended to be limiting in any way.

### EXAMPLES

### Example 1

Amorphous Memantine Hydrochloride Memantine hydrochloride (2 g) was completely dissolved in water (40 ml) in a round bottom flask to obtain a clear solution. The solution was then transferred to a heavy walled lyophilization flask (2 liters). The solution in flask is rapidly cooled by liquid nitrogen until it is frozen. The lyophilizer was evacuated and maintained under about 0.01 mm Hg vacuum for about 5 hours. The residue was submitted for powder X-ray analysis, which produced a featureless diffractogram (FIG. 1).

### Example 2

Amorphous Memantine Hydrochloride Memantine Hydrochloride (1.2 g) was dissolved in methanol (40 ml) at ambient temperature to obtain a clear solution. The solvent was evaporated under vacuum (80 mm Hg) at about 20 to about 50° C. Drying was continued under vacuum at about 60° C. to about 80° C. for about two hour. Similar to Example 1 above, the powder X-ray diffractograrn of the solid (FIG. 1) showed that the resulting substance was in amorphous form.

### Example 3

Amorphous Memantine Hydrochloride Memantine Hydrochloride (1.0 g) was dissolved in methanol (50 ml) at ambient temperature to obtain a clear solution. The solvent was evaporated under vacuum (80 mm Hg) at about 20 to about 60° C. Drying was continued under vacuum at about 60° C. to about 90° C. for about two hour. Similar to Example 1 above, the powder X-ray diffractograrn of the solid (FIG. 1) showed that the resulting substance was in amorphous form.

1. A novel amorphous form of 1-amino-3, 5-dimethyltricyclo-[3,3,1,1^{3,7}]decane hydrochloride (memantine hydrochloride).

2. The amorphous form of memantine hydrochloride according to claim 1, characterized by an X-ray powder diffraction pattern substantially in accordance with FIG. 1.

3. The amorphous form of memantine hydrochloride according to claim 1 containing less than about 10% crystalline memantine hydrochloride.

4. The amorphous form of memantine hydrochloride according to claim 1 essentially free of crystalline memantine hydrochloride.

5. A process for preparing amorphous form of memantine hydrochloride comprising the steps of dissolving memantine hydrochloride in a solvent to form a solution, and removing the solvent from the solution to afford amorphous form of memantine hydrochloride.

6. The process of claim 5 wherein the solvent is removed by lyophilization.

7. The process of claim 6 wherein the solvent is an aqueous solvent.

8. The process of claim 7 wherein the aqueous solvent is water.

9. The process of claim 6 wherein the solvent is a C₁-C₄ alcohol.

10. The process of claim 5 wherein the solvent is removed by distillation.

11. The process of claim 10 wherein the solvent is an aqueous solvent.

12. The process of claim 10 wherein the solvent is a C₁-C₄ alcohol.

13. The process of claim 12 wherein the C₁-C₄ alcohol is methanol or ethanol.

14. The process of 10 wherein the distillation is performed at a pressure of about 400 mm Hg or less.

15. The process of claim 14 wherein the distillation is performed at a pressure of about 80 mm Hg or less.

16. The process of claim 10 wherein the distillation is performed at a pressure of from about 30 to about 80 mm Hg.

17. A pharmaceutical composition comprising an amorphous form of 1-amino-3, 5-dimethyltricyclo[3,3,1,1^{3,7}]decane hydrochloride (memantine hydrochloride) and pharmaceutically acceptable carrier, diluent, excipient, additive, filler, lubricant, solvent, binder or stabilizer.

18. A pharmaceutical composition according to claim 17, in the form of a tablet, troche, powder, syrup, patch, liposome, injection, dispersion, suspension, solutions, capsule, cream, oitment or aerosol.

19. A method for the treating or preventing cerebral ischemia after apoplexy, open-heart surgery, cardiac standill, subarachnoidal hemorrhage, transient cerebro-ischemic attacks, perinatal asphyxia, anoxia, hypoglycemia, apnoca and Alzheimer's disease or conditions in which N-methyl-D-aspartate (NMDA)-receptor antagonist is implicated, including a long-term non-ischemic neurodegenerative disease, comprising administrating an effective amount of an amorphous form of memantine hydrochloride according to any one of claims 1 to 4 and a pharmaceutically acceptable carrier, diluent, excipient, binder, additive, filler, lubricant, solvent or stabilizer to a patient in need thereof.
